(19) United States
(12) Patent Application Publication
(10) Pub. No.: US 2021/0293822 A1
(43) Pub. Date: Sep. 23, 2021

(54) METHODS FOR PREDICTING THE SURVIVAL TIME OF PATIENTS SUFFERING FROM A MICROSATELLITE UNSTABLE CANCER

(71) Applicants: INSERM (INSTITUT NATIONAL DE LA SANTE ET DE LA RECHERCHE MEDICALE), Paris (FR); ASSISTANCE PUBLIQUE - HOPITAUX DE PARIS, Paris (FR); LIGUE NATIONALE CONTRE LE CANCER, Paris (FR); SORBONNE UNIVERSITE, Paris (FR)

(72) Inventors: Alex DUVAL, Paris (FR); Thierry ANDRE, Paris (FR); Magali SVRCEK, Paris (FR); Aurelien DE REYNIES, Paris (FR); Laetitia MARISA, Paris (FR)

(21) Appl. No.: 17/221,171

(22) Filed: Apr. 2, 2021

Related U.S. Application Data
(62) Division of application No. 16/066,949, filed on Jun. 28, 2018, now abandoned.

(30) Foreign Application Priority Data
Dec. 29, 2015 (EP) 15307157.6

(51) Int. Cl. G01N 33/574

(52) U.S. Cl. CPC G01N 33/57419; G01N 2800/56; G01N 2800/52; G01N 2333/90241

(57) ABSTRACT

The present invention relates to methods for predicting the survival time of patients suffering from a micro satellite unstable cancer. In particular, the present invention relates to a method for predicting the survival time of a patient suffering from a micro satellite unstable cancer comprising i) determining the expression level of at least one gene encoding for an immune checkpoint protein in a tumor tissue sample obtained from the patient, ii) comparing the expression level determined at step i) with a predetermined reference value and iii) concluding that the patient will have a long survival time when the level determined at step i) is lower than the predetermined reference value or concluding that the patient will have a short survival time when the level determined at step i) is higher than the predetermined reference value.

### FIELD OF THE INVENTION

The present invention relates to methods for predicting the survival time of patients suffering from a microsatellite unstable cancer.

### BACKGROUND OF THE INVENTION

The MSI phenotype (also called mutator phenotype) is associated with a broad spectrum of both inherited and sporadic malignancies. All these tumors share analogous underlying mechanisms that are MSI-driven and lead the cell to undergo malignant transformation following the accumulation of somatic mutational events, notably in cancer-related genes containing coding repeated sequences. All MSI tumors are more or less highly immunogenic with increased expression of immune checkpoint molecules in the cancer core. Consequently, it is expected that immune checkpoint overexpression may constitute a theranostic predictor associated with bad survival in MSI cancer overall regardless of primary tumor location.

The normal function of the mismatch repair (MMR) system is to recognize and repair the errors that arise during DNA replication, as well as to repair some forms of DNA damage. MMR deficiency leads to the development of tumors (8-9), mainly colorectal cancers (CRCs), through a distinctive molecular pathway characterized by the genetic instability of microsatellite repeat sequences (MSI, Microsatellite Instability) throughout the genome (10). This MSI-driven pathway to cancer results in numerous frameshifts that lead to the synthesis of aberrant potentially immunogenic neo-antigens by the tumor cells (for review, see 13-14). Probably as a consequence, MSI tumors are highly infiltrated with cytotoxic T-cell lymphocytes (CTL) expressing activation markers and Th1 cells, and several publications reported the density of this infiltrate should constitute a main cause for the improved prognosis of MSI CRCs compared to Microsatellite Stable (MSS) CRC (4-6). On the other hand, recent findings also highlighted the concomitant and specific overexpression of multiple active checkpoints counterbalancing the active Th1/CTL microenvironment in MSI colorectal carcinoma and protecting these tumors from killing, e.g. -CTLA-4, PD-1, PD-L1, and LAG-3—currently targeted by immunotherapy (15). In line with this, Le et al. (16) evaluated the clinical activity of an anti-PD-1 immune checkpoint inhibitor (pembrolizumab) in a cohort of patients with metastatic carcinoma displaying or not MSI due to MMR-deficiency. Results from this phase 2 study convincingly showed that MSI status was likely to predict clinical benefit of immune checkpoint blockade with this agent, i.e. objective response rate of 40% (4 of 10 patients) compared to 0% (0 of 18 patients) for patients with MSS metastatic CRC. Predicting optimal immunotherapy with one or several agents accurately requires the identification and validation of reliable biomarkers.

### SUMMARY OF THE INVENTION

The present invention relates to methods for predicting the survival time of patients suffering from a microsatellite unstable cancer. In particular, the present invention is defined by the claims.

### DETAILED DESCRIPTION OF THE INVENTION

High infiltration with cytotoxic T-cell lymphocytes (CTL) as well as activated Th1 cells has been reported to constitute a main cause for the improved prognosis of colorectal cancer (CRC) displaying microsatellite instability (MSI) (4-6). However, recent findings also highlighted this active CTL/Th1 microenvironment was counterbalanced by up-regulated expression of multiple immune checkpoints in these tumors (15) with clinical benefit of immune checkpoint blockade in metastatic MSI CRC patients (16). Here the inventors evaluated the putative prognostic value of immune checkpoints in MSI cancers, particularly MSI CRC taking into account their CTL/Th1 microenvironment. They analyzed the expression of 19 transcripts encoding immune-modulator or -checkpoints together with 15 CTL/Th1/cytotoxicity markers in two independent multicentric series of stage I-IV primary CRC totaling 232 MSI and 971 MSS CRC. They confirmed these molecules were generally overexpressed in MSI compared to MSS colon tumors and non-tumoral colorectal mucosa. Overexpression of several checkpoints was associated with a poorer prognosis independently from tumor stage and despite concomitant high expression levels of CTL/Th1/cytotoxicity markers. The inventors demonstrated that the metagenes corresponding to ICKs, CTL, cytotoxicity and Th1 orientation were overexpressed in MSI tumors demonstrating their prognostic value. Functional investigations confirmed the negative impact of ICKs expression on the proliferation of in-filtrating CD8 T cells in MSI neoplasms. These findings suggest that immune checkpoints, and in particular the druggable PD-1, PD-L1, LAG-3, TIM-3, and IDO molecules, have a dominant impact above other immune components for prognosing MSI cancers such as MSI CRC, highlighting their relevance as therapeutic targets and theranostic biomarkers in these tumors.

Accordingly the first object of the present invention relates to a method for predicting the survival time of a patient suffering from a microsatellite unstable cancer comprising i) determining the expression level of at least one gene encoding for an immune checkpoint protein in a tumor tissue sample obtained from the patient, ii) comparing the expression level determined at step i) with a predetermined reference value and iii) concluding that the patient will have a long survival time when the level determined at step i) is lower than the predetermined reference value or concluding that the patient will have a short survival time when the level determined at step i) is higher than the predetermined reference value.

As used herein, the term “microsatellite unstable cancer” has its general meaning in the art and refers to cancer liable to have a MSI phenotype. “A cancer liable to have a MSI phenotype” refers to a sporadic or hereditary cancer in which microsatellite instability may be present (MSI, Microsatellite Instability) or absent (MSS, Microsatellite Stability). Detecting whether microsatellite instability is present may for example be performed by genotyping microsatellite markers, such as BAT25, BAT26, NR21, NR24 and NR27, e.g. as described in Buhard et al., J Clin Oncol 24 (2), 241 (2006) and in European patent application No. EP 11 305 160.1. A cancer is defined as having a MSI phenotype if instability is detected in at least 2 microsatellite markers. On the contrary, if instability is detected in one or no microsatellite marker, then said cancer has a MSS phenotype. A sporadic cancer liable to have a MSI phenotype may refer to a cancer due to somatic genetic alteration of one of the Mismatch Repair (MMR) genes MLH1, MSH2, MSH6 and PMS2. For example, a sporadic cancer liable to have a MSI phenotype can be a cancer due to de novo bi-allelic methylation of the promoter of MLH1 gene. An hereditary cancer liable to have a MSI phenotype may refer to a cancer that occurs in the context of Lynch syndrome or Constitutional Mismatch-Repair Deficiency (CMMR-D). A patient suffering from Lynch syndrome is defined as a patient with an autosomal mutation in one of the 4 genes MLH1, MSH2, MSH6, and PMS2. A patient suffering from CMMR-D is defined as a patient with a germline biallelic mutation in one of the 4 genes MLH1, MSH2, MSH6, and PMS2. The MSI phenotype is present across different cancer types such as described in Ronald J Hause et al., Nat. Med 2016 (39). Accordingly, the term “microsatellite unstable cancer” refers to any cancer type having MSI phenotype. Examples of cancers liable to have a MSI phenotype include adenoma or primary tumors, such as colorectal cancer (also called colon cancer or large bowel cancer), colon adenocarcinoma, rectal adenocarcinoma, gastric cancer, stomach cancer, endometrial cancer, uterine cancer, uterine corpus endometrial carcinoma, breast cancer, bladder cancer, hepatobiliary tract cancer, liver hepatocellular carcinoma, urinary tract cancer, urothelial carcinoma, ovary cancer, ovarian serous cystadenocarcinoma, lung adenocarcinoma, lung squamous cell carcinoma, bladder cancer, prostate cancer, kidney cancer, kidney renal papillary cell carcinoma, head and neck cancer, skin cancer, skin cutaneous melanoma, thyroid carcinoma, squamous cell carcinoma, lymphomas, leukemia, brain cancer, brain lower grade glioma, glioblastoma, glioblastoma multiforme, astrocytoma, neuroblastoma and cancers described in Ronald J Hause et al., Nat. Med 2016 (39).

In some embodiments, the patient suffers from a microsatellite unstable colorectal cancer.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum). Determination of MSI status in CRC involves routine methods well known in the art.

In some embodiments, the microsatellite unstable cancer is at Stage I, II, III, or IV as determined by the TNM classification, but however the present invention is accurately useful for predicting the survival time of patients when said cancer has been classified as Stage II or III by the TNM classification, i.e. non metastatic cancer.

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumour of the patient or performed in metastatic sample distant from the primary tumor of the patient. For example an endoscopical biopsy performed in the bowel of the patient suffering from the colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, and through methods of gene or protein expression analysis, including genomic and proteomic analysis. The tumor tissue sample can be subjected to a variety of well-known post-collection preparative and storage techniques (e.g., fixation, storage, freezing, etc.) prior to determining the expression level of the gene of interest. Typically the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides). The tumour tissue sample can be used in microarrays, called as tissue microarrays (TMAs). TMA consist of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA. Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Examples of genes encoding for a immune checkpoint inhibitor thus include IDO1, CD40, CD274, ICOS, TNFRSF9, TNFRSF18, LAG3, IL2RB, HAVCR2, TNFRSF4, CD276, CTLA4, PDCD1LG2, VTCN1, PDCD1, BTLA, CD28, C10orf54 and CD27 (see Table A). In the present specification, the name of each of the genes of interest refers to the internationally recognised name of the corresponding gene, as found in internationally recognised gene sequences and protein sequences databases, in particular in the database from the HUGO Gene Nomenclature Committee, that is available notably at the following Internet address: http://www.gene.ucl.ac.uk/nomenclature/index.html. In the present specification, the name of each of the various biological markers of interest may also refer to the internationally recognised name of the corresponding gene, as found in the internationally recognised gene sequences and protein sequences databases ENTRE ID, Genbank, TrEMBL or ENSEMBL. Through these internationally recognised sequence databases, the nucleic acid sequences corresponding to each of the gene of interest described herein may be retrieved by the one skilled in the art.

TABLE A
Examples of genes encoding for immune checkpoint
proteins:

| Gene | Name | GENE ID |
|---|---|---|
| IDO1 | indoleamine 2,3-dioxygenase 1 | 3620 |
| CD40 | CD40 molecule, TNF receptor | 958 |
| | superfamily member 5 | |
| CD274 | CD274 molecule, also known as | 29126 |
| | B7-H; B7H1; PDL1; PD-L1; | |
| | PDCD1L1; PDCD1LG1 | |
| ICOS | inducible T-cell co-stimulator | 29851 |
| TNFRSF9 | tumor necrosis factor receptor | 3604 |
| | superfamily member 9, also | |
| | known as ILA; 4-1BB; CD137; | |
| | CDw137 | |
| TNFRSF18 | tumor necrosis factor receptor | 8784 |
| | superfamily member 18, also | |
| | known as AITR; GITR; CD357; | |
| | GITR-D | |
| LAG3 | lymphocyte-activation gene 3 | 3902 |
| IL2RB | interleukin 2 receptor, beta | 3560 |
| HAVCR2 | hepatitis A virus cellular | 84868 |
| | receptor 2 | |
| TNFRSF4 | tumor necrosis factor receptor | 7293 |
| | superfamily member 4 | |
| CD276 | CD276 molecule | 80381 |
| CTLA4 | cytotoxic T-lymphocyte- | 1493 |
| | associated protein 4 | |
| PDCD1LG2 | programmed cell death 1 ligand | 80380 |
| | 2, also known as B7DC; Btdc; | |
| | PDL2; CD273; PD-L2; | |
| | PDCD1L2; bA574F11.2 | |
| VTCN1 | V-set domain containing T cell | 79679 |
| | activation inhibitor 1, also | |
| | known as B7H4 | |
| PDCD1 | programmed cell death 1, also | 5133 |
| | known as PD1; PD-1; CD279; | |
| | SLEB2; hPD-1; hPD-1; hSLE1 | |
| BTLA | B and T lymphocyte associated | 151888 |
| CD28 | CD28 molecule | 940 |
| C10orf54 | chromosome 10 open reading | 64115 |
| | frame 54 | |
| CD27 | CD27 molecule | 939 |

In some embodiments, the method of the present invention comprises determining the expression level of at least one gene (i.e. 1, 2, 3, 4, 5, 6, 7, 8, 9, 10, 11, 12, 13, 14, 15, 16, 17, 18, or 19 genes) selected from the group consisting of IDO1, CD40, CD274, ICOS, TNFRSF9, TNFRSF18, LAG3, IL2RB, HAVCR2, TNFRSF4, CD276, CTLA4, PDCD1LG2, VTCN1, PDCD1, BTLA, CD28, C10orf54 and CD27.

In some embodiments, the method of the present invention comprises determining the expression level of at least one gene (i.e. 1, 2, 3, 4, 5, 6, 7, 8, 9, 10, or 11 genes) encoding for inhibitory immune checkpoint protein selected from the group consisting of IDO1, CD274, LAG3, HAVCR2, CD276, CTLA4, PDCD1LG2, VTCN1, PDCD1, BTLA and C10orf54.

In some embodiments, the method of the present invention comprises determining the expression level of at least one gene (i.e. 1, 2, 3, 4, 5, 6, 7, and 8 genes) encoding for stimulatory immune checkpoint protein selected from the group consisting of CD40, ICOS, TNFRSF9, TNFRSF18, IL2RB, TNFRSF4, CD28, and CD27.

In some embodiments, the method of the present invention comprises determining the expression level of at least one gene encoding for inhibitory immune checkpoint protein selected from the group consisting of IDO1, CD274, LAG3, HAVCR2, CD276, CTLA4, PDCD1LG2, VTCN1, PDCD1, BTLA and C10orf54 in combination with at least one gene encoding for stimulatory immune checkpoint protein selected from the group consisting of CD40, ICOS, TNFRSF9, TNFRSF18, IL2RB, TNFRSF4, CD28, and CD27.

As used herein the term “cytotoxic T-cell lymphocytes marker” or “CTLs” has its general meaning in the art and refers to markers of tumor-infiltrating T cells or cytotoxic T-cell lymphocytes. The term “cytotoxic T-cell lymphocytes marker” also refers to markers of immune activation of cytotoxic T cells associated with immune anti-tumoral response (16, 24).

In some embodiments, the method of the present invention further comprises i) determining the expression level of at least one gene encoding for a cytotoxic T-cell lymphocytes marker, cytotoxicity marker or Th1 orientation marker, ii) comparing the expression level determined at step i) with a predetermined reference value and iii) concluding that the patient will have a long survival time when the level determined at step i) is higher than the predetermined reference value or concluding that the patient will have a short survival time when the level determined at step i) is lower than the predetermined reference value.

As used herein the term “cytotoxicity marker” has its general meaning in the art and refers to cytotoxicity-related genes associated with immune anti-tumoral response (16, 24).

As used herein the term “Th1 orientation marker” has its general meaning in the art and refers to T helper 1 cells (Th1 cell) factors associated with immune anti-tumoral response (16, 24).

In some embodiments, the method comprises determining the expression level of at least one gene encoding for an immune checkpoint protein in combination with at least one gene encoding for a cytotoxic T-cell lymphocytes (CTL) marker selected from the group consisting of CD3G, CD3E, CD3D, PTPRC and CD8A.

In some embodiments, the method of the invention comprises determining the expression level of at least one gene encoding for an immune checkpoint protein in combination with at least one gene encoding for a cytotoxicity marker selected from the group consisting of PRF1, GZMH, GNLY, GZMB, GZMK and GZMA.

In some embodiments, the method of the invention comprises determining the expression level of at least one gene encoding for an immune checkpoint protein in combination with at least one gene encoding for a Th1 orientation marker selected from the group consisting of TBX21 and IFNG.

In some embodiments, the method of the present invention comprises determining the expression of 1, 2, 3, 4, 5, 6, 7, 8, 9, 10, 11, 12, 13, 14, 15, 16, 17, 18, or 19 genes selected from the group consisting of IDO1, CD40, CD274, ICOS, TNFRSF9, TNFRSF18, LAG3, IL2RB, HAVCR2, TNFRSF4, CD276, CTLA4, PDCD1LG2, VTCN1, PDCD1, BTLA, CD28, C10orf54 and CD27 in combination with 1, 2, 3, 4, 5, 6, 7, 8, 9, 10, 11, 12, or 13 genes selected from the group consisting of CD3G, CD3E, CD3D, PTPRC, CD8A, PRF1, GZMH, GNLY, GZMB, GZMK, GZMA, TBX21 and IFNG.

In some embodiments, the expression level of a gene is determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-overlapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute level or normalized level. Typically, levels are normalized by correcting the absolute level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the level in one sample, e.g., a subject sample, to another sample, or between samples from different sources.

In some embodiments, the expression level of a gene is determined by determining the quantity of the protein translated from said gene. Methods for quantifying protein of interest are well known in the art and typically involve immunohistochemistry. Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the protein of interest, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated with the binding partners having for the protein of interest. After washing, the labeled antibodies that are bound to the protein of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems.

For example, one or more labels can be attached to the antibody, thereby permitting detection of the target protein (i.e the immune checkpoint protein; cytotoxic T-cell lymphocytes marker; cytotoxicity marker; or Th1 orientation marker). Exemplary labels include radioactive isotopes, fluorophores, ligands, chemiluminescent agents, enzymes, and combinations thereof. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In some embodiments, the label is a quantum dot. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled.

In some embodiments, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the protein in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantify the presence of the specified biomarker (i.e. immune checkpoint protein). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the immune checkpoint protein) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining of the sample, and represents the amount of target biomarker (e.g., the immune checkpoint protein) present in the sample. Optical density or percentage area values can be given a scaled score, for example on an integer scale.

Thus, in some embodiments, the method of the present invention comprises the steps consisting in i) providing one or more immunostained slices of tissue section obtained by an automated slide-staining system by using a binding partner capable of selectively interacting with the protein of interest (e.g. an antibody as above described), ii) proceeding to digitalisation of the slides of step i) by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity or the absolute number of stained cells in each unit.

Multiplex tissue analysis techniques might also be useful for quantifying several proteins of interest in the tumor tissue sample. Such techniques should permit at least five, or at least ten or more biomarkers to be measured from a single tumor tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In some embodiments, the L-IHC method can be performed on any of a variety of tissue samples, whether fresh or preserved. The samples included core needle biopsies that were routinely fixed in 10% normal buffered formalin and processed in the pathology department. Standard five μη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μη thick coated polymer backbone with 0.4μη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of the gene in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the gene in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples; b) providing, for each ample provided at step a), information relating to the actual clinical outcome for the corresponding subject (i.e. the duration of the survival); c) providing a serial of arbitrary quantification values; d) determining the expression level of the gene for each sample contained in the collection provided at step a); e) classifying said samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the subjects from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example the expression level of the gene has been assessed for 100 samples of 100 subjects. The 100 samples are ranked according to the expression level of the gene. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of the gene corresponding to the boundary between both subsets for which the p value is minimum is considered as the predetermined reference value.

It should be noted that the predetermined reference value is not necessarily the median value of expression levels of the gene. Thus in some embodiments, the predetermined reference value thus allows discrimination between a poor and a good prognosis for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of the gene with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the expression level of the gene, where values higher than 5 reveal a poor prognosis and values less than 5 reveal a good prognosis. In some embodiments, a subject may be assessed by comparing values obtained by measuring the expression level of the gene and comparing the values on a scale, where values above the range of 4-6 indicate a poor prognosis and values below the range of 4-6 indicate a good prognosis, with values falling within the range of 4-6 indicating an intermediate occurrence (or prognosis).

The method of the present invention is also suitable for determining whether a patient suffering from a microsatellite unstable cancer is eligible for a treatment with an immune checkpoint inhibitor.

Thus a further object of the present invention relates to a method for determining whether a patient suffering from a microsatellite unstable cancer will achieve a response with an immune checkpoint inhibitor comprising i) determining the expression level of at least one gene encoding for an immune checkpoint protein in a tumor tissue sample obtained from the patient, ii) comparing the expression level determined at step i) with a predetermined reference value and iii) concluding that the patient will achieve a response when the level determined at step i) is higher than the predetermined reference value.

In some embodiments, patient suffers from microsatellite unstable colorectal cancer.

In a further aspect, the method of the invention further comprises i) determining the expression level of at least one gene encoding for a cytotoxic T-cell lymphocytes marker, cytotoxicity marker or Th1 orientation marker, ii) comparing the expression level determined at step i) with a predetermined reference value and iii) concluding that the patient will achieve a response when the level determined at step i) is higher than the predetermined reference value.

The method is thus particularly suitable for discriminating responder from non responder. As used herein the term “responder” in the context of the present disclosure refers to a patient that will achieve a response, i.e. a patient where the cancer is eradicated, reduced or improved. According to the invention, the responders have an objective response and therefore the term does not encompass patients having a stabilized cancer such that the disease is not progressing after the immune checkpoint therapy. A non-responder or refractory patient includes patients for whom the cancer does not show reduction or improvement after the immune checkpoint therapy. According to the invention the term “non responder” also includes patients having a stabilized cancer. Typically, the characterization of the patient as a responder or non-responder can be performed by reference to a standard or a training set. The standard may be the profile of a patient who is known to be a responder or non responder or alternatively may be a numerical value. Such predetermined standards may be provided in any suitable form, such as a printed list or diagram, computer software program, or other media. When it is concluded that the patient is a non responder, the physician could take the decision to stop the immune checkpoint therapy to avoid any further adverse sides effects.

As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future.

The immune checkpoint inhibitors include peptides, antibodies, nucleic acid molecules and small molecules. In particular, the immune checkpoint inhibitor of the present invention will enhance the cytotoxic activity of CD8 T cells. As used herein “CD8 T cells” has its general meaning in the art and refers to a subset of T cells which express CD8 on their surface. They are MHC class I-restricted, and function as cytotoxic T cells. “CD8 T cells” are also called CD8 T cells are called cytotoxic T lymphocytes (CTL), T-killer cell, cytolytic T cells, CD8+ T cells or killer T cells. CD8 antigens are members of the immunoglobulin supergene family and are associative recognition elements in major histocompatibility complex class I-restricted interactions. The ability of the immune checkpoint inhibitor to enhance T CD8 cell killing activity may be determined by any assay well known in the art. Typically said assay is an in vitro assay wherein CD8 T cells are brought into contact with target cells (e.g. target cells that are recognized and/or lysed by CD8 T cells). For example, the immune checkpoint inhibitor of the present invention can be selected for the ability to increase specific lysis by CD8 T cells by more than about 20%, preferably with at least about 30%, at least about 40%, at least about 50%, or more of the specific lysis obtained at the same effector: target cell ratio with CD8 T cells or CD8 T cell lines that are contacted by the immune checkpoint inhibitor of the present invention, Examples of protocols for classical cytotoxicity assays are conventional.

In some embodiments, the immune checkpoint inhibitor is an antibody selected from the group consisting of anti-CTLA4 antibodies (e.g. Ipilimumab), anti-PD1 antibodies, anti-PDL1 antibodies, anti-TIM-3 antibodies, anti-LAG3 antibodies, anti-B7H3 antibodies, anti-B7H4 antibodies, anti-BTLA antibodies, and anti-B7H6 antibodies.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human monoclonal antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

A further aspect, the invention relates to a method for treating microsatellite unstable cancer in a patient in need thereof comprising the steps of: a) determining whether the patient suffering from a microsatellite unstable cancer will achieve a response with an immune checkpoint inhibitor by performing the method according to the invention, and b) administering the immune checkpoint inhibitor, if said patient has been considered as a responder.

In some embodiments, the patient suffers from microsatellite unstable colorectal cancer.

In some embodiments, the immune checkpoint inhibitor of the present invention is administered to the patient in combination with chemotherapy.

As used herein “chemotherapy” has its general meaning in the art and is a cancer treatment that uses drugs to stop the growth of cancer cells, either by killing the cells or by stopping them from dividing. The said drug can be for example a small molecule: small molecules which can be conveniently used for the invention include in particular genotoxic drugs. Preferentially, genotoxic drugs used for cancer treatment such as colorectal cancer treatment include busulfan, bendamustine, carboplatin, carmustine, chlorambucil, cisplatin, cyclophosphamide, dacarbazine, daunorubicin, doxorubicin, epirubicin, etoposide, idarubicin, ifosfamide, irinotecan (and its active metabolite sn38), lomustine, mechlorethamine, melphalan, mitomycin c, mitoxantrone, oxaliplatin, temozolamide and topotecan. Even more preferentially, the genotoxic drugs according to the invention are oxaliplatin, irinotecan, and irinotecan active metabolite sn38. However, the invention should not be understood as being limited to genotoxic drugs, as many other types of small molecules can also be used in the context of this invention. For example, antimetabolites such as 5-FU (and its prodrug capecitabine), tegafur-uracil (or UFT or UFUR), leucovorin (LV, folinic acid), or proteasome inhibitors such as bortezomib are also encompassed by the scope of this invention.

In some embodiments, when it is concluded that the patient will not achieve a response with an immune checkpoint inhibitor, it can be decide that the patient will be treated only with chemotherapy.

The invention will be further illustrated by the following figures and examples. However, these examples and figures should not be interpreted in any way as limiting the scope of the present invention.

### FIGURES

FIG. 1A-1E. Prognostic value of immune gene expression. A. Overall survival stratified by MSI/MSS status (left) and CMS subtypes (right). Curves of overall survival (OS) rate (y-axis) according to time from diagnosis (in years) (x-axis) were obtained by the method of Kaplan and Meier for both the CIT and TCGA series. Differences between survival distributions were assessed by the log-rank test using an end point of 5 years. B. Prognostic values of immune gene/metagene expression and of clinical factors in MSI tumors. Forest plot of overall survival (OS) hazard ratios (HR) estimated by combining independent univariate Cox analyses on the CIT and TCGA series, adjusted for TNM stage. HR, as well as related Wald test p-value and 95% confidence intervals (95% C.I.), are given for metagenes (which aggregates the gene expression values of a gene set related to the four immune categories (immune checkpoints (ICK), cytotoxic T lymphocytes (CTL), cytotoxicity, Th1 functional orientation), individual immune genes and clinical annotations. Diamonds represent the HR and horizontal bars the 95% C.I. Red indicates a HR >1 with p-value <0.1 (worse prognosis), blue a HR <1 with p-value <0.1 (better prognosis) and grey a HR with Wald test p-value ≤0.1. C Overall survival stratified by overexpression of immune checkpoint genes within MSI (left), MSS (middle) and both CRC (right). MSI tumors in the higher quartile of ICK metagene values, in CIT and TCGA series independently, were assigned ICK+ (n=55), and the other tumors ICK− (n=139). The minimal ICK metagene value within MSI ICK+ tumors was used to divide MSS tumors into ICK+ (n=26) and ICK− (n=765). Curves of overall survival (OS) rate (y-axis) according to time from diagnosis (in years) (x-axis) were obtained by the method of Kaplan and Meier for both the CIT and TCGA series. Differences between survival distributions were assessed by the log-rank test using an end point of 5 years. D Prognostic value of immune and Immunoscore® gene expression metagenes according to CRC subtypes. Heatmap of univariate Cox analysis p-values of immune and Immunoscore® metagenes, adjusted for TNM stage, colored by significance and HR sign (red for worse prognosis (HR >1) and blue for better prognosis (HR <1)). Analyses were performed independently on the CIT and TCGA series and further combined, within all, MSI, MSS and MSS subdivided according to CMS subtypes tumors. Cox analysis HR and p-values are indicated in each cell. n corresponds to the number of samples evaluated. IS-like v1 and v2 correspond to metagenes of genes used in the 2 main versions of the Immunoscore® from Galon and colleagues. E Bivariate Cox models of OS, combining the ICK metagene with other metagenes, in MSI tumors. Forest plot of OS HR estimated by bivariate Cox analysis of ICK, CTL, CY-TOX, Th1 and Immunoscore®-like metagene expression in the CIT series, adjusted by TNM stage. The expression of metagenes related to CTL/Th1/Cytotoxicity/Immunoscore® markers was associated with trends for better prognosis (Hazard Ratio (HR) <1), whereas the ICK metagene was associated with worse prognosis (HR >1) in all bivariate models. Strikingly, whatever the association with prognosis (negative/positive/none) of ICK and CTL/Th1/Cytotoxicity/Immunoscore®-related metagenes, a similar pattern was found within different CRC subgroups (MSI and MSS CMS). This suggests a substantial correlation between these signals within each CRC stratum.

FIG. 2A-2C. Prognostic value of immune checkpoints in an independent metastatic MSI CRC patient series A. Prognostic value of ICK gene expression in metastatic MSI tumors. Forest plot of hazard ratio (HR) estimated by univariate Cox analysis of overall survival (OS, left panel) and survival after relapse (SAR, right panel) on all ICK genes available in the NanoString data. Diamonds represent HR estimates and bars the related 95% confidence intervals. Red indicates a worse prognosis hazard ratio, blue a better prognosis and grey a HR Wald test p-value ≤0.1. B. Prognostic value of metagene expression in MSI metastatic tumors. Forest plot of HR estimated by univariate Cox analysis of survival after relapse for metagene expression. The ICK metagene aggregates the 3 most associated ICK genes in univariate analysis (HAVCR2, CD274 and LAG3). The other metagene aggregate genes available in the NanoString dataset for the corresponding category were: CTL=CD3D, CD3G, CD8A, PTPRC; CYTOX=GNLY, GZMK; Th1=IFNG. Metagene expression values were calculated by averaging the expression values obtained within the corresponding gene set. C. Bivariate Cox models of OS and SAR, combining the ICK metagene with other metagenes, in metastatic MSI tumors. Forest plot of SAR HR estimated by bivariate Cox analysis of ICK, CTL, CYTOX, Th1 and Immunoscore®-like metagene expression in the independent MSI metastatic series.

FIG. 3. Bivariate Cox models for analysing the impact of stimulatory/Inhibitory ICK expression on the survival of MSI CRC patients. In bivariate Cox models, the expression of stimulatory ICK metagene was not associated with bad prognosis, as expected, whereas this remains to be the case with the expression of inhibitory ICK metagene.

### EXAMPLE

Materials and Methods

Immune Genes

Immune checkpoint and modulator genes were selected according to Llosa et al. (15) and a recent review (23). Markers for cytotoxic T lymphocytes, cytotoxicity and T helper1 were selected as described earlier (15, 24).

Cohort Data

Tissue samples from a large, multisite cohort of CRC patients were collected as part of the ‘Cartes d'Identité des Tumeurs’ (CIT) research program/network, including tumors with or without microsatellite instability (MSI or MSS respectively) and adjacent non-tumoral tissue samples (NT). Samples from 146 MSI, 444 MSS tumors and 56 NT were analyzed for gene expression profiling on Affymetrix U133 plus 2 chips as described earlier (25). Data were normalized using frozen RMA method (26) followed by a Combat normalization (27) to remove technical batch effects (SVA R package). For validation purposes, the CRC cohort from the TCGA consortium was used. Both datasets were centered for each gene by subtracting the median value of the non-tumoral sample. To obtain a summarized value for each immune gene category, a metagene value was computed by taking the median value of all genes in the category per sample.

A retrospective, additional multisite series of 28 stage 4 primary MSI CRC was analyzed as an independent study for gene expression using NanoString technology on a set of immune genes that included 14 of the 32 analyzed markers. All patients from this metastatic cohort (11 synchronous metastatic lesions, 17 metachronous metastatic lesions) received standard of care chemotherapy but did not benefit from ICK blockade. The Nanostring data set also includes a subset of the CIT cohort.

Associations between gene expression and survival were assessed by univariate and bivariate Cox proportional-hazards regression analyses using the R package survival.

Immune Genes

We investigated 32 immune markers classified into four gene groups: (i) immune checkpoints and modulators (n=19; CD40, CD274, ICOS, LAG3, IL2RB, HAVCR2, TNFRSF4/9/18, CD276, CTLA4, PDCD1LG2, VTCN1, PDCD1, BTLA, CD28, C10orf54, CD27, IDO1); (ii) cytotoxicity (n=6; GZMA/B/K/H, GLNY, PRF1); (iii) Th1 orientation (n=2; TBX21, IFNG); and (iv) cytotoxic lymphocytes (n=5; CD8A, CD3D/E/G, PTPRC) (for review, see (24)).

TCGA Cohort

For validation purposes, the CRC cohort from the TCGA consortium was used. Preprocessed gene expression RNA-seq data were downloaded at the Broad Institute TCGA Genome Data Analysis Center (2015): Firehose stddata_2015_06_01 run. Broad Institute of MIT and Harvard. doi:10.7908/C1251HBG. Data were combined and normalized according to TCGA RNA-seq pipeline using RSEM quantification. The dataset contained 86 MSI, 527 MSS and 51 NT samples.

Survival Analysis

Associations between gene expression and survival were assessed by univariate and bivariate Cox proportional-hazards regression analyses using the R package survival. All Cox models were stratified by TNM stage and, for the CIT cohort, by clinical centers. For the CIT and TCGA cohorts, overall survival was used as the end point and was defined as the time from surgery to death (any cause) of the patient, or to last contact. The delays were censored at 5 years. Survival annotations were available for 137 MSI and 439 MSS CRC patients in the CIT cohort, and for 57 MSI and 352 MSS CRC patients in the TCGA cohort.

Separate analyses were performed independently on both data sets. Results from these two series were combined using a meta-analysis approach from DerSimonian et al. (38) using the inverse variance method for pooling of survival data, implemented in the R package meta (function metagen). For the metastatic patient cohort, survival after relapse was used. This was defined as the time from metastasis diagnosis to death from any cause, or last contact with the patient.

Functional Analysis

An enrichment analysis was performed to evaluate pathways associated with overexpression of ICKs using MSigDB gene sets. Significant genes associated with ICK overexpression were selected by a moderated t-test between low and high ICK expression level in MSI tumors (the top and bottom 30 samples based on the ICK metagene). The top 100 to 500 significant genes were evaluated for gene set enrichments by hypergeometric tests. The median pvalue across gene selections was used to select significant gene sets. Only a selection amongst the significant gene sets, based on functional interest, was shown.

The abundance of immune cell populations was estimated using MCP-counter software (28).

Immunohistochemistry and ImmunoFluorescence Procedures

20 FFPE tumor samples of MSI colon cancers were sliced in thin tissue sections of 4 μm.

For IHC, automated routine staining procedures were carried out for HE, PD-L1 (Ventana, SP142) and CD8 (Dako, M7103) using Ventana Benchmark. Relative quantification for PD-L1 staining was performed independently by two pathologists. Absolute CD8 quantification was carried out with Definiens Tissue Studio software. Briefly, after numeration using Nanozoomer 2.0HT and NDPscan software (both from Hamamatsu), slides for each sample were processed and analyzed in several areas that were manually defined by a pathologist. Screen captures were made with NDPview software (Hamamatsu). PD-L1 staining without nuclei counterstaining was also performed and merged with HE staining using Paint.net free software (dotPDN LLC).

For IF procedures, the same samples from MSI patients were stained using Ki67-Alexa-Fluor 488 labelled (BD Pharmingen, 558616, dilution 1/10 incubated overnight) and CD8 (Dako, M7103, dilution 1/100 during one hour) antibodies. Secondary goat-anti-mouse Alexa-Fluor 555 was also used (Life Technologies, A21422, dilution 1/500 during 30 minutes) for CD8 staining (see also Supplementary Materials and Methods). Slides were then mounted using DAPI-containing mounting medium (Sigma, F6057), kept at 4° C. and imaged the following day using spectral microscopy technology (Mantra Workstation, PerkinElmer) at X20 magnification. DAPI-only positive cells, Ki67-only positive cells, CD8-only positive cells and CD8/Ki67 double positive cells were phenotyped using a trainable algorithm from inForm software (PerkinElmer).

Results

Prognostic Value of Immune Genes and Metagenes in Function of CMS Classification of Colorectal Cancer

To test our working hypothesis, we evaluated the prognostic significance of ICKs, Th1, CTLs and cytotoxicity markers in the combined CIT (n=590 CRC, comprising 146 MSI and 444 MSS) and TCGA (n=613 CRC, comprising 86 MSI and 527 MSS) series. In both cohorts, MSS tumors were categorized into one of the four CMS of CRC (22). We investigated 32 immune markers classified into the above four gene groups (for review, see (24)). Four of the 19 immune checkpoints and modulators were not found significantly overexpressed in CRC as compared to non-tumor colonic mucosa (NT), and were subsequently removed. Further analyses were thus carried on the 28 remaining genes. Four metagenes were then built from the four gene groups, by aggregating the corresponding genes (median of log 2 expression fold changes, relative to NT). Finally, in order to obtain Immunoscore® surrogates, six Immunoscore®-like metagenes were built based on the expression of Immunoscore® related markers (Table 1).

TABLE 1
description of the six Immunoscore ®-like metagenes

| Name | Genes involved |
|---|---|
| Immunoscore ®-like VO | CD3, CD8A |
| Immunoscore ®-like V1 | CD3, CD8A, PTPRC |
| Immunoscore ®-like V2 | CD8A, PTPRC, GZMB, MS4A1 |
| Immunoscore ®-like V3 | CD8A, PTPRC, GZMB, MS4A1, CD68 |
| Immunoscore ®-like V4 | CD3, CD8A, PTPRC, GZMB, MS4A1 |
| Immunoscore ®-like V5 | CD3, CD8A, PTPRC, GZMB, MS4A1, CD68 |

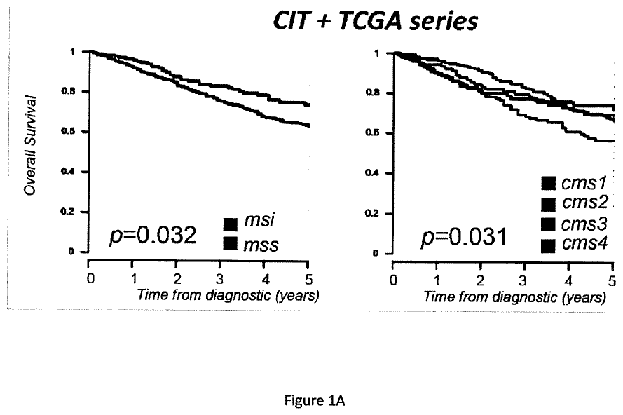

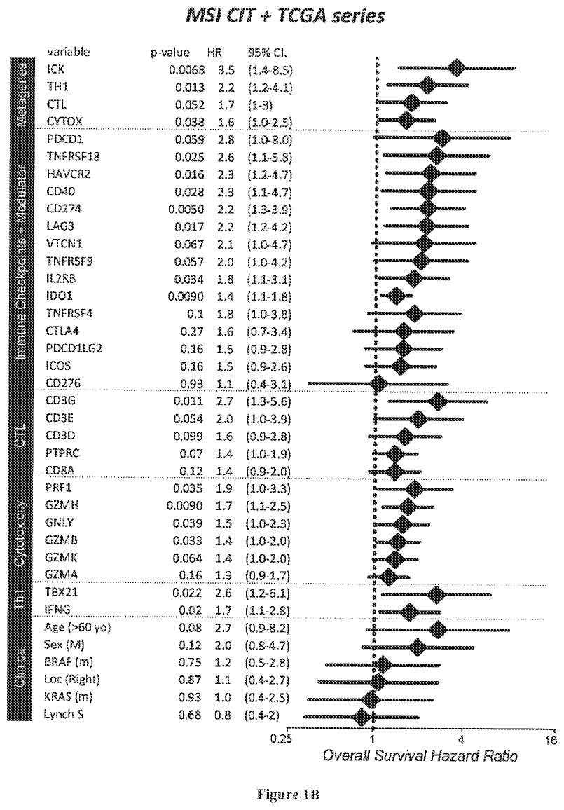

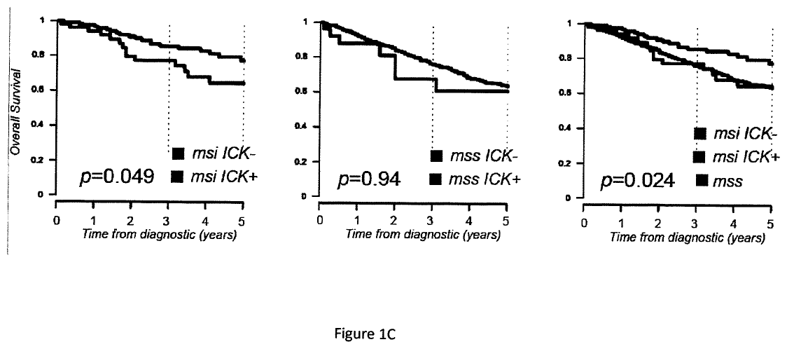

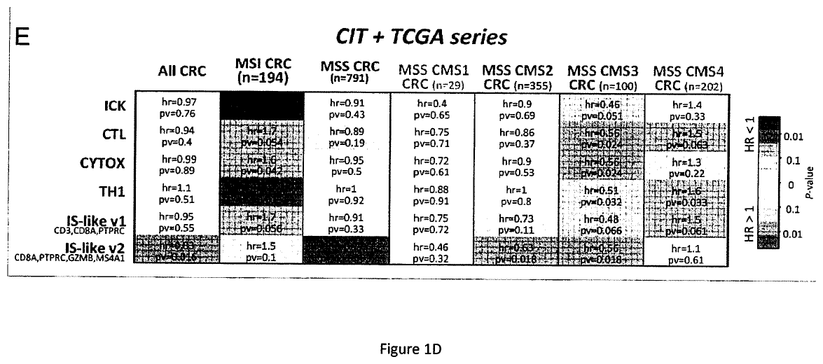

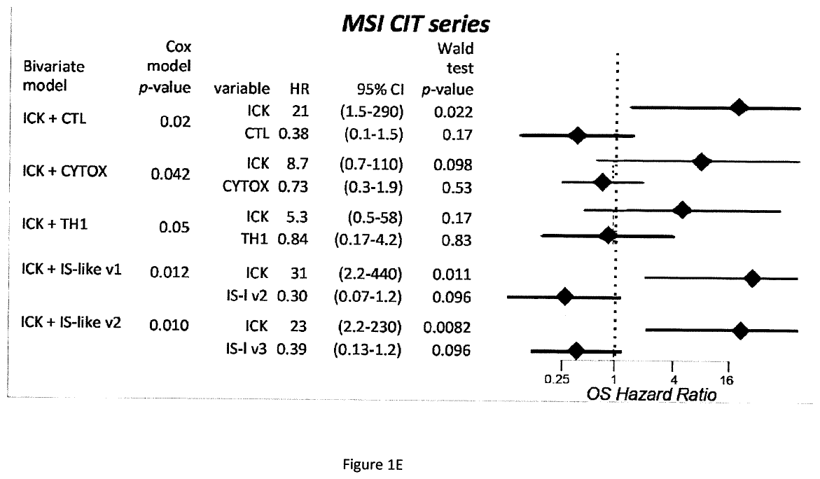

As a preliminary step, univariate Cox models of overall survival (OS) were used to analyze the prognostic values of MSI and CMS status after adjusting for stage and tumor series. As expected, these models showed an improved prognosis for patients with MSI CRC compared to those with MSS CRC, as well as significant prognostic value for the CMS classification (FIG. 1A). Univariate Cox models were then used to analyze the 28 immune markers mentioned above after adjusting for stage and series. Most ICK genes were individually associated with poorer prognosis in MSI CRC, as reflected by the ICK metagene (FIGS. 1B-D). This association remained significant in non-metastatic MSI CRC. No prognostic association was observed in patients with MSS CRC, either for individual ICK markers or for ICK metagene (FIGS. 1C-D). Subdividing MSS CRC by CMS did not change this result, except in CMS3, where ICK metagene was found associated to better prognosis (FIG. 1D). As expected, the expression of most Immunoscore®-like metagenes were associated with improved outcome of CRC patients (FIG. 1D). However, when the CMS and MSI/MSS status was taken into account, expression of these Immunoscore® surrogates was predictive of better prognosis only in MSS tumors from CMS2 and CMS3. In contrast, they had no prognostic relevance in CRC from CMS1 and CMS4 (FIG. 1D).

In univariate models, the overexpression of CTL/Th1/cytotoxicity/Immunoscore® markers and metagenes was also associated with adverse prognosis in MSI CRC (FIGS. 1B and 1D). We therefore hypothesized that high expression levels of ICKs in MSI CRC could counterbalance and mask the expected positive effect of CTL/Th1/cytotoxic cells or Immunoscore®-related cells on prognosis. Bivariate Cox models at the metagene level were consistent with this assumption (FIG. 1E).

All together, these data underline that ICKs and Immunoscore® biomarkers constitute independent prognostic factor for overall survival in MSI and MSS tumor, respectively.

Expression and Prognostic Value of Immune Checkpoints in an Independent Metastatic MSI CRC Patient Series

The CIT and TCGA series included mostly non-metastatic MSI CRC patients (n=220/232, 94.8%). Since ICK blockade was recently proposed as a promising new therapy for metastatic MSI CRC, we endeavored to further evaluate the prognostic relevance of ICK expression in an independent cohort of stage 4 MSI CRC. To do this, we analyzed the expression of 7 ICKs (CD274, PDCD1LG2, HAVCR2, LAG3, ICOS, CTLA4, PDCD1) using NanoString technology in a retrospective, multisite series comprised of 28 stage 4 primary MSI CRC treated with standard care.

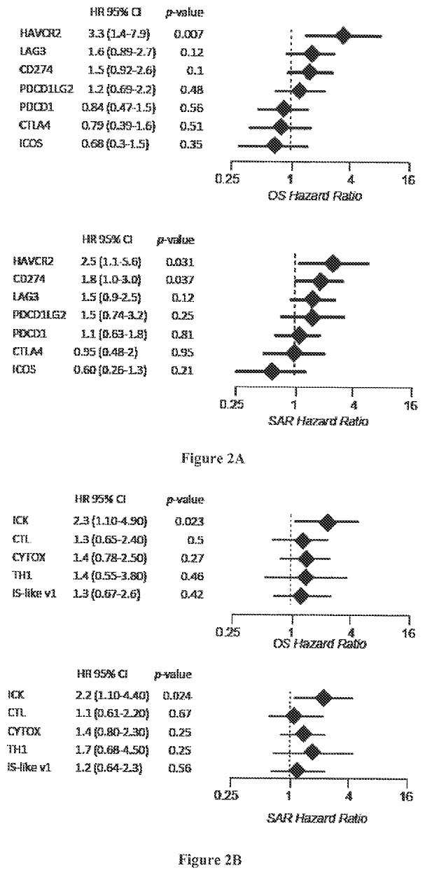

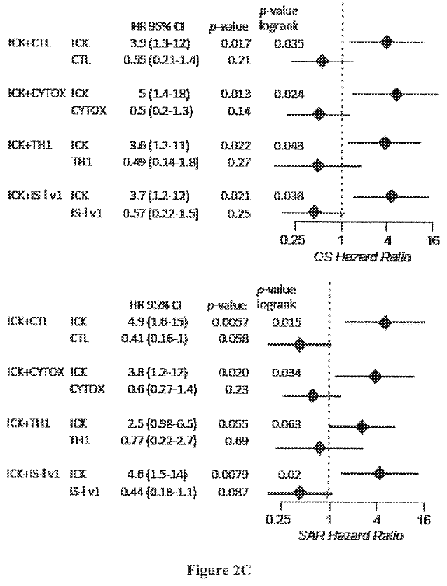

As with non-metastatic MSI colon tumors, we observed significant association of PD-L1 (CD274), TIM-3 (HAVCR2) and LAG3 expression with worse OS and worse survival after re-lapse (SAR) (FIG. 2A). Again, the overexpression of metagenes corresponding to CTL/Th1/Cytotoxicity/Immunoscore® markers (CTL=CD3D, CD3G, CD8A, PTPRC; CYTOX=GNLY, GZMK; Th1=IFNG) tended to associate with adverse prognosis in univariate models (FIG. 2B). However, in bivariate Cox models, the expression of CTL/Th1/Cytotoxicity/Immunoscore®-related metagenes was associated with good prognosis (Hazard Ratio (HR) <1), as expected (FIG. 2C). Several ICK markers, in particular the druggable PD-L1 (CD274) and TIM-3 (HAVCR2) molecules (23), are therefore likely to constitute biomarkers for poor prognosis in both metastatic and non-metastatic MSI CRC patients.

Impact of Stimulatory/Inhibitory ICK Expression on the Survival of MSI CRC Patients

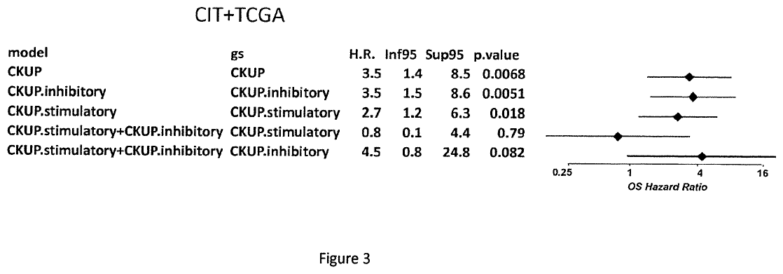

We performed bivariate Cox models for analysing the impact of stimulatory/Inhibitory ICK expression on the survival of MSI CRC patients (FIG. 3). In bivariate Cox models, the expression of stimulatory ICK metagene was not associated with bad prognosis, as expected, whereas this remains to be the case with the expression of inhibitory ICK metagene (FIG. 3).

Immune Checkpoint Gene Expression Distribution in Colorectal Cancer

We next investigated the level of variation in ICK expression amongst CRC tumors in order to further assess their relevance as prognostic and theranostic markers. ICK expression was analyzed in stage 1-4 CRC and in non-tumor colonic mucosa (NT) from our CIT cohort (590 CRC, 56 NT) and in the TCGA cohort (613 CRC, 51 NT). In both cohorts, the metagenes corresponding to ICKs, CTL, cytotoxicity and Th1 orientation were overexpressed in MSI and in MSS tumors belonging to CMS1 and CMS4 as compared to MSS CRC from CMS2 and CMS3. Variable expression of ICKs relative to NT was noted in all CMS subtypes in both cohorts. A high degree of heterogeneity was found in CMS1 tumors, particularly in MSI tumors where high to very high expression levels of ICKs was observed in a large proportion of cases.

Expression levels for all of the 28 immune markers were highly correlated in the MSI CRC from both cohorts. The present results highlight the extent of heterogeneity of MSI CRC with respect to immunity and to the overexpression of ICK molecules. This was observed regardless of MSI CRC origin (inherited or sporadic) or of other clinical or molecular parameters such as gender, tumor location, tumor stage, CMS, or KRAS/BRAF mutations. Considerable variation in the expression of ICK markers was also observed in the independent metastatic MSI CRC series evaluated by Nanostring.

Functional Relevance of Immune Checkpoint Expression in CRC

We next addressed the possible physiological relevance of ICK overexpression in CRC. Tumor infiltration by immune cells was quantified using MCP-counter software (28) in both the CIT and TCGA cohorts. A strong correlation was observed between ICK expression and infiltration by lymphoid (NK, T cells, cytotoxic cells) and myeloid cells. In contrast, B cells, fibroblasts, vessels and granulocytes were less associated with ICK expression or not at all. These results suggest that ICK expression occurs in response to an efficient in situ adaptive T cell immune response. Pathway enrichment analysis (hypergeometric tests) using MSigDB pathways was performed to compare the expression profiles of MSI tumors with low vs high ICK expression levels. Significant associations were observed between ICK expression and immune response gene sets, including positive activation of T cell response, negative regulation of T cell activation, T cell exhaustion, IL-10 response and chronic viral infection (29). Hence, we conclude there is a strong correlation between ICK expression and the presence of an exhausted T cell immune response in MSI CRC.

To further investigate the functional relevance of ICKs in MSI tumors, we studied 8 primary MSI tumors showing up-regulation of ICKs and 12 without. PD-L1 and CD8 expression were examined using immunohistochemistry (IHC). PD-L1 expression was observed only in the tumor bed, whereas CD8 was present both in the tumor core and in stromal areas. Moreover, PD-L1 expression correlated strongly with ICK expression, while CD8 infiltrates in both the tumor bed and in peritumoral stroma also correlated with PD-L1 IHC staining. Proliferation and functional activity of CD8 T cells were then determined using multi-parametric immunofluorescence microscopy. CD8 T cells that were close to or in contact with PD-L1-expressing tumors were less proliferative, as observed with Ki67 labeling. These results indicate that interactions between CD8 T cells and ICK ligands in MSI primary tumors can impede CD8 T cell function.

Discussion:

During cancer progression, tumor-infiltrating T cells have been shown to display increased, chronic expression of different antagonist ICKs including PD-1, LAG-3, and TIM-3, causing functional exhaustion and unresponsiveness of T cells (30). The exhausted CD8 T cells fail to proliferate in response to antigen and lack critical anticancer effector functions such as cytotoxicity and Interferon gamma cytokine secretion (31). These observations have provided the rationale to develop antibodies that target these regulatory molecules. So called checkpoint inhibitors could boost the anticancer immune response and the potential relevance of these inhibitors for the treatment of metastatic MSI CRC patients was highlighted in a recent publication (16). In the present study we showed that ICK overexpression represents a more accurate prognostic biomarker for MSI CRC patients treated with standard care than the classical assessment of T cell number by Immunoscore® (1). This may be explained by the presence of exhausted non-proliferative CD8 T cells in the core of these neoplasms. More generally, our data indicates that assessment of the prognostic significance of antitumor immunity in CRC needs to take into account ICK expression. This is particularly relevant for colon tumors displaying immunogenic profiles with both high Immunoscore® and ICK expression, such as in MSI tumors and probably a significant proportion of MSS CRC.

The current results were obtained using univariate Cox models for survival analysis and a transcriptome-based method to quantify both ICK and CTL/Th1/Cytotoxicity (Immunoscore®) markers in tumor and tumor-adjacent normal mucosa samples. We validated our method by building Immunoscore®-like surrogates that were associated with significantly improved survival of CRC patients. Nervertheless, under the same conditions, the CTL/Th1/Cytotoxicity and Immunoscore® markers were both associated with worse prognostic in MSI CRC from both the CIT and TCGA series. These results are potentially in conflict with a recent publication that observed significant association between Immunoscore®, as assessed by Immuno-histochemistry and Immunomorphometry, and improved outcome in a single series of 105 MSI CRC patients (17). Although the studies are not directly comparable, here we assessed three independent cohorts of CRC patients totaling more than 1,200 cases and including 260 MSI CRC. It does not include classical Immunoscore® evaluation by immunohistochemistry. However, we performed bivariate Cox analysis at the metagene level. This revealed that expression of metagenes related to CTL/Th1/Cytotoxicity and Immunoscore® markers was associated with trends for better prognosis in MSI CRC from both the CIT and TCGA series, whereas the ICK metagene was significantly associated with worse prognosis. In contrast with the earlier study that focused only on PD1/PDL1 couple (17), a more global assessment of ICK gene expression in the tumor core, as proposed in the present study, allows a more holistic view of the T cell immune response in CRC. The transcriptome based method reported here is easier to use in both research and clinical settings, and more amenable to standardization. Importantly, it can be also used to test publicly available clinical data sets, whereas this is not possible with Immunoscore® because of the need to assess primary tumor samples.

The development of monoclonal Antibodies that target checkpoints inhibitors is an exciting new development in cancer therapy. Recent clinical trials have demonstrated that antibodies targeting PD-1 or PD-L1 can induce major response in many types of cancers (32). The overall survival rate with more than 5 years follow up for stage 2 and 3 MSI CRC patients is approximately 70% without adjuvant chemotherapy and 75-90% with standard care adjuvant chemotherapy (33-35). However, the 5-year survival rate for stage 4 MSI CRC patients is less than 5% 33. We report here for the first time the prognostic significance of ICK overexpression in both metastatic and non-metastatic MSI CRC and in the absence of immunotherapy. These findings should help to better inform the prognosis of MSI CRC patients and to identify those who are at high risk of relapse. They may be useful for guiding future immunotherapy involving antibody blockade of ICKs in non-metastatic MSI CRC patients and to have predictive factors of immunotherapy efficacy for patients with metastatic disease.

To conclude, our results highlight the extent of heterogeneity of CRC with respect to immunity and the overexpression of ICK molecules in particular. They suggest that prediction of CRC patient outcomes through evaluation of immune components in the tumor microenvironment will likely be improved by the integration of ICK markers, the prognosis of colon tumors being determined by the CTL/ICK balance. More particularly, our results indicate that ICK expression impacts the prognosis of MSI tumors and that overexpression of these molecules impedes CD8 T cell function in MSI CRC, regardless of their CMS subgroup. To inform future immunotherapy involving antibody blockade of ICKs and resistance to these molecules in MSI CRC patients, additional studies on the molecular mechanisms underlying the immune reaction against MSI tumor cells are required. These mechanisms may depend on the number and type of MSI-driven mutational events that drive tumor progression and lead to the synthesis of aberrant, immunogenic peptides (36), thereby impacting the relation of tumor cells with their complex immune microenvironment including ICK expression and/or function. Identifying these somatic events and investigating their functional relevance with respect to quantitative and qualitative anti-tumoral immunity may improve the personalized treatment of MSI CRC patients with ICK inhibitors, in both metastatic and non-metastatic settings.

### REFERENCES

Throughout this application, various references describe the state of the art to which this invention pertains. The disclosures of these references are hereby incorporated by reference into the present disclosure.
1. Galon J, Costes A, Sanchez-Cabo F, et al. Type, density, and location of immune cells within human colorectal tumors predict clinical outcome. Science. 2006; 313(5795):1960-4.
2. Angell H K, Gray N, Womack C, Pritchard D I, Wilkinson R W, Cumberbatch M. Digital pattern recognition-based image analysis quantifies immune infiltrates in distinct tissue regions of colorectal cancer and identifies a metastatic phenotype. Br J Cancer. 2013; 109(6):1618-24.
3. Mlecnik B, Tosolini M, Kirilovsky A, et al. Histopathologic-based prognostic factors of colorectal cancers are associated with the state of the local immune reaction. J Clin Oncol. 2011; 29(6):610-8.
4. Lothe R A, Peltomaki P, Meling G I, et al. Genomic instability in colorectal cancer: relationship to clinicopathological variables and family history. Cancer Res. 1993; 53 (24):5849-52.
5. Gryfe R, Kim H, Hsieh E T, et al. Tumor microsatellite instability and clinical outcome in young patients with colorectal cancer. N Engl J Med. 2000; 342(2):69-77.
6. Bauer K, Nelius N, Reuschenbach M, et al. T cell responses against microsatellite instability-induced frameshift peptides and influence of regulatory T cells in colorectal cancer. Cancer Immunol Immunother. 2013; 62(1):27-37.
7. Aaltonen L A, Peltomaki P, Leach F S, et al. Clues to the pathogenesis of familial colorectal cancer. Science. 1993; 260(5109):812-6.
8. Leach F S, Nicolaides N C, Papadopoulos N, et al. Mutations of a mutS homolog in hereditary nonpolyposis colorectal cancer. Cell. 1993; 75(6):1215-25.
9. Thibodeau S N, Bren G, Schaid D. Microsatellite instability in cancer of the proximal colon. Science. 1993; 260(5109):816-9.
10. Ionov Y, Peinado M A, Malkhosyan S, Shibata D, Perucho M. Ubiquitous somatic mutations in simple repeated sequences reveal a new mechanism for colonic carcinogenesis. Nature. 1993; 363(6429):558-61.
11. Fishel R, Lescoe M K, Rao M R, et al. The human mutator gene homolog MSH2 and its association with hereditary nonpolyposis colon cancer. Cell. 1993; 75(5):1027-38.
12. Dorard C, de Thonel A, Collura A, et al. Expression of a mutant HSP110 sensitizes colorectal cancer cells to chemotherapy and improves disease prognosis. Nat Med. 2011; 17:1283-89.
13. Duval A, Hamelin R. Mutations at coding repeat sequences in mismatch repair-deficient human cancers: toward a new concept of target genes for instability. Cancer Res. 2002; 62(9):2447-54.
14. Hamelin R, Chalastanis A, Colas C, et al. [Clinical and molecular consequences of microsatellite instability in human cancers]. Bull Cancer. 2008; 95(1):121-32.
15. Llosa N J, Cruise M, Tam A, et al. The vigorous immune microenvironment of microsatellite instable colon cancer is balanced by multiple counter-inhibitory checkpoints. Cancer Discov. 2015; 5(1):43-51.
16. Le D T, Uram J N, Wang H, et al. PD-1 Blockade in Tumors with Mismatch-Repair Deficiency. N Engl J Med. 2015; 372(26):2509-20.
17. Mlecnik B, Bindea G, Angell H K, et al. Integrative Analyses of Colorectal Cancer Show Immunoscore Is a Stronger Predictor of Patient Survival Than Microsatellite Instability. Immunity. 2016; 44(3): 698-711.
18. Dunne P D, McArt D G, O'Reilly P G, et al Immune-Derived PD-L1 Gene Expression Defines a Subgroup of Stage II/III Colorectal Cancer Patients with Favorable Prognosis Who May Be Harmed by Adjuvant Chemotherapy. Cancer Immunol Res. 2016; 4(7):582-91.
19. Masugi Y, Nishihara R, Yang J, et al. Tumour CD274 (PD-L1) expression and T cells in colorectal cancer. Gut. 2016.
20. Lee L H, Cavalcanti M S, Segal N H, et al. Patterns and prognostic relevance of PD-1 and PD-L1 expression in colorectal carcinoma. Mod Pathol. 2016.
21. Li Y, Liang L, Dai W, et al. Prognostic impact of programed cell death-1 (PD-1) and PD-ligand 1 (PD-L1) expression in cancer cells and tumor infiltrating lymphocytes in colorectal cancer. Mol Cancer. 2016; 15(1):55.
22. Guinney J, Dienstmann R, Wang X, et al. The consensus molecular subtypes of colorectal cancer. Nat Med. 2015; 21(11):1350-6.
23. Melero I, Berman D M, Aznar M A, Korman A J, Perez Gracia J L, Haanen J. Evolving synergistic combinations of targeted immunotherapies to combat cancer. Nat Rev Cancer. 2015; 15(8):457-72.
24. Fridman W H, Pages F, Sautes-Fridman C, Galon J. The immune contexture in human tumours: impact on clinical outcome. Nat Rev Cancer. 2012; 12(4):298-306.
25. Marisa L, de Reynies A, Duval A, et al. Gene expression classification of colon cancer into molecular subtypes: characterization, validation, and prognostic value. PLoS Med. 2013; 10(5):e1001453.
26. McCall M N, Bolstad B M, Irizarry R A. Frozen robust multiarray analysis (fRMA). Biostatistics. 2010; 11(2):242-53.
27. Johnson W E, Li C, Rabinovic A. Adjusting batch effects in microarray expression data using empirical Bayes methods. Biostatistics. 2007; 8(1):118-27.
28. Becht E, Giraldo N A, Lacroix L, et al. Estimating the population abundance of tissue-infiltrating immune and stromal cell populations using gene expression. Genome Biol. 2016; 17(1):218.
29. Wherry E J, Kurachi M. Molecular and cellular insights into T cell exhaustion. Nat Rev Immunol. 2015; 15(8):486-99.
30. Blackburn S D, Shin H, Haining W N, et al. Coregulation of CD8+ T cell exhaustion by multiple inhibitory receptors during chronic viral infection. Nat Immunol. 2009; 10(1):29-37.
31. Ferris R L, Lu B, Kane L P. Too much of a good thing? Tim-3 and TCR signaling in T cell exhaustion. J Immunol. 2014; 193(4):1525-30.
32. Topalian S L, Drake C G, Pardoll D M. Immune checkpoint blockade: a common denominator approach to cancer therapy. Cancer Cell. 2015; 27(4):450-61.
33. Sargent D J, Marsoni S, Monges G, et al. Defective mismatch repair as a predictive marker for lack of efficacy of fluorouracil-based adjuvant therapy in colon cancer. J Clin Oncol. 2010; 28(20):3219-26.
34. Venderbosch S, Nagtegaal I D, Maughan T S, et al. Mismatch repair status and BRAF mutation status in metastatic colorectal cancer patients: a pooled analysis of the CAIRO, CAIRO2, COIN, and FOCUS studies. Clin Cancer Res. 2014; 20(20):5322-30.
35. Andre T, de Gramont A, Vernerey D, et al. Adjuvant Fluorouracil, Leucovorin, and Oxaliplatin in Stage II to III Colon Cancer: Updated 10-Year Survival and Outcomes According to BRAF Mutation and Mismatch Repair Status of the MOSAIC Study. J Clin Oncol. 2015; 33(35):4176-87.
36. Saeterdal I, Bjorheim J, Lislerud K, et al. Frameshift-mutation-derived peptides as tumor-specific antigens in inherited and spontaneous colorectal cancer. Proc Natl Acad Sci USA. 2001; 98(23):13255-60.
37. Becht E, de Reynies A, Giraldo N A, et al Immune and Stromal Classification of Colorectal Cancer Is Associated with Molecular Subtypes and Relevant for Precision Immunotherapy. Clin Cancer Res. 2016; 22(16):4057-66.
38. DerSimonian R, Laird N. Meta-analysis in clinical trials. Control Clin Trials. 1986; 7(3):177-88.
39. Hause R J, Pritchard C C, Shendure J, Salipante S J. Classification and characterization of microsatellite instability across 18 cancer types. Nat Med. 2016 November; 22(11):1342-1350.

1. A method for treating a non-metastatic microsatellite unstable cancer in a patient in need thereof comprising the steps of:
obtaining at least one non-metastatic microsatellite unstable cancer cell from the patient,
measuring an expression level of at least one gene encoding an immune checkpoint protein in the at least one non-metastatic microsatellite unstable cancer cell, wherein the at least one gene includes VTCN1,
comparing the expression level of the at least one gene encoding an immune checkpoint protein with a predetermined reference value, wherein the predetermined reference value is a corresponding expression level obtained from microsatellite stable cancer cells or non-tumoral cells,
determining that the patient will respond to an immune checkpoint inhibitor when the expression level is higher than the predetermined reference value, or
determining that the patient will not respond to an immune checkpoint inhibitor when the expression level is lower than the predetermined reference value, and
treating the patient with the immune checkpoint inhibitor when the expression level is higher than the predetermined reference value, or
treating the patient with chemotherapy when the expression level is lower than the predetermined reference value.

2. The method of claim 1, wherein the microsatellite unstable cancer is at Stage I, II, III, or IV as determined by the TNM classification.

3. The method of claim 1, wherein the microsatellite unstable cancer is microsatellite unstable colorectal cancer.

4. The method of claim 1, wherein the at least one gene further comprises at least one gene selected from the group consisting of IDO1, CD40, CD274, ICOS, TNFRSF9, TNFRSF18, LAG3, IL2RB, HAVCR2, TNFRSF4, CD276, CTLA4, PDCD1LG2, and PDCD1.

5. The method of claim 1, wherein the immune checkpoint inhibitor is at least one antibody selected from the group consisting of anti-CTLA4, anti-PD1, anti-PDL1, anti-TIM-3, anti-LAG3, anti-B7H3, anti-B7H4, anti-BTLA, and anti-B7H6.

6. A method for identifying and treating a patient who has a non-metastatic microsatellite unstable cancer with an immune checkpoint inhibitor treatment, comprising the steps of:
obtaining a tumor tissue sample of the non-metastatic microsatellite unstable cancer from the patient,
measuring in the tumor tissue sample an increased expression level of at least one gene encoding an immune checkpoint protein in the at least one non-metastatic microsatellite unstable cancer cell as compared to a predetermined reference value, wherein the at least one gene includes VTCN1 and wherein the predetermined reference value is a corresponding expression level obtained from microsatellite stable cancer cells or non-tumoral cells, and
treating the patient with the immune checkpoint inhibitor.

7. The method of claim 6, wherein the microsatellite unstable cancer is at Stage I, II, III, or IV as determined by the TNM classification.

8. The method of claim 6, wherein the microsatellite unstable cancer is microsatellite unstable colorectal cancer.

9. The method of claim 6, wherein the at least one gene further comprises at least one gene selected from the group consisting of IDO1, CD40, CD274, ICOS, TNFRSF9, TNFRSF18, LAG3, IL2RB, HAVCR2, TNFRSF4, CD276, CTLA4, PDCD1LG2, and PDCD1.

10. The method of claim 6, wherein the immune checkpoint inhibitor is at least one antibody selected from the group consisting of anti-CTLA4, anti-PD1, anti-PDL1, anti-TIM-3, anti-LAG3, anti-B7H3, anti-B7H4, anti-BTLA, and anti-B7H6.

11. A method for identifying and treating a patient suffering from an exhausted T cell response to a non-metastatic microsatellite cancer that predisposes the patient to having a relapse of the microsatellite unstable cancer, comprising the steps of:
obtaining a tumor core sample of the non-metastatic microsatellite unstable cancer from the patient,
detecting infiltration by lymphoid and myeloid cells in the tumor core sample,
measuring in the tumor core sample an expression level of at least one gene encoding an immune checkpoint protein in the at least one non-metastatic microsatellite unstable cancer cell, wherein the at least one gene includes VTCN1,
comparing the expression level of the at least one gene measured to that of a predetermined reference value, wherein the predetermined reference value is a corresponding expression level obtained from microsatellite stable cancer cells or non-tumoral cells,
determining that the patient will respond to an immune checkpoint inhibitor when the expression level is higher than the predetermined reference value, or
determining that the patient will not respond to an immune checkpoint inhibitor when the expression level is lower than the predetermined reference value, and
treating the patient with the immune checkpoint inhibitor in a quantity sufficient to stimulate a T cell immune response when the expression level is higher than the predetermined reference value, or
treating the patient with chemotherapy when the expression level is lower than the predetermined reference value.

12. The method of claim 11, wherein the microsatellite unstable cancer is at Stage I, II, III, or IV as determined by the TNM classification.

13. The method of claim 11, wherein the microsatellite unstable cancer is microsatellite unstable colorectal cancer.

14. The method of claim 11, wherein the at least one gene further comprises at least one gene selected from the group consisting of IDO1, CD40, CD274, ICOS, TNFRSF9, TNFRSF18, LAG3, IL2RB, HAVCR2, TNFRSF4, CD276, CTLA4, PDCD1LG2, and PDCD1.

15. The method of claim 11, wherein the immune checkpoint inhibitor is at least one antibody selected from the group consisting of anti-CTLA4, anti-PD1, anti-PDL1, anti-TIM-3, anti-LAG3, anti-B7H3, anti-B7H4, anti-BTLA, and anti-B7H6.
